(19) United States
(12) Patent Application Publication
(10) Pub. No.: US 2022/0389411 A1
(43) Pub. Date: Dec. 8, 2022

(54) METHOD AND SYSTEM FOR INTEGRATING MORPHOLOGICAL CHARACTERISTICS AND GENE EXPRESSION OF SINGLE-CELL

(71) Applicant: WELLSIM BIOMEDICAL TECHNOLOGIES, INC, Rodeo, CA (US)

(72) Inventor: YUCHAO CHEN, Rodeo, CA (US)

(21) Appl. No.: 17/548,790

(22) Filed: Dec. 13, 2021

Related U.S. Application Data
(63) Continuation-in-part of application No. PCT/IB2021/060684, filed on Nov. 18, 2021.
(60) Provisional application No. 63/197,331, filed on Jun. 5, 2021.

(51) Int. Cl. C12N 15/10; B01L 3/00

(52) U.S. Cl. CPC C12N 15/1072; B01L 3/502715; B01L 3/502761; C12N 15/1096; B01L 2200/0652; B01L 2400/0424

(57) ABSTRACT

The present application provides a method and a system for integrating morphological characteristics and gene expression of individual cells. The method comprises the following steps: providing a microfluidic device, which comprises a microwell array and an interdigital electrode, and each microwell comprises a plurality of capture oligonucleotides; injecting cells into the microwells, capturing a single cell and recording morphological characteristics of the cell; lysing the cell so that the mRNA released by the cell is captured by the capture oligonucleotide; reverse transcribing the captured mRNA to obtain cDNA; performing a PCR amplification reaction on the cDNA to obtain a cDNA library and sequencing the cDNA library; reading the cell barcode sequence and the unique molecular identifier sequence according to sequencing results, and the morphological characteristics and gene expression of the cell in the microwell are integrated together.

### FIELD

The present application relates to biotechnologies, and more particularly, to a method and a system for integrating morphological characteristics and gene expression of individual cells.

### BACKGROUND

Single-cell RNA sequencing (scRNA-seq) technologies have revolutionized the way for transcriptomic analysis of multicellular tissues. Via investigation of thousands of cells at single-particle resolution, scRNA-seq can provide quantitative expression profiles of individual cells with valuable insights into cellular differences, such as cell types and cell states, which are usually elusive in the traditional bulk RNA-seq analysis. The distinct advantages of single-cell transcriptomics enable multi-dimensional investigation of individual cells to, for example, decipher tumor heterogeneity, reveal complex and rare cell populations, and uncover regulatory relationships between genes, offering a strong basis for designing precision medicine and targeted therapy.

On the other hand, due to the rapid evolution of computational technologies (e.g., deep learning and neural networks), cellular morphological profiling based on high-throughput and high-content image processing has become an emerging tool for various biological and medical applications. For example, imaging flow cytometry (IFC), the representative technology in this field, can extract hundreds of morphological features (e.g., shape, size, intensity, and texture) from each individual cell with AI-driven algorithms, allowing classification of complex cell phenotypes, identification of rare cells, and discovery of useful targets for disease diagnosis, personalized medicine, and drug development.

Since the morphological characteristics are highly correlated with gene expression patterns, researchers have shown substantial interests in the integration of phenomics and transcriptomics data for novel biological insights. However, currently there is no such an approach that could link gene expression profiles to morphological phenotypes at single-cell level with a high-throughput manner. Some studies are carried out based on bulk RNA-seq analysis while ignore cellular heterogeneity. The others rely on manual collection of each target cell using pipette, which have a limited throughput.

### BRIEF DESCRIPTION OF THE DRAWINGS

Implementations of the present disclosure will now be described, by way of embodiments only, with reference to the attached figures.

FIG. 1 is a flowchart for the method for integrating morphological characteristics and gene expression of individual cells.

FIG. 2 is a diagrammatic view of a microfluidic device according to an embodiment of the present application.

FIG. 3 is a cross-sectional view taken along A-A in FIG. 2.

FIG. 4 is a diagrammatic view of a capture oligonucleotide according to an embodiment of the present application.

FIG. 5 is a diagrammatic view of a process of synthesizing capture oligonucleotides via inkjet printing.

FIG. 6 is a diagrammatic view of the capture oligonucleotide synthesized in FIG. 5.

FIG. 7 is a diagrammatic view of a process for performing transcription analysis on cells according to an embodiment of the application.

FIG. 8 is a diagrammatic view illustrating morphological features linking to gene expression profiles at single-cell level with a high throughput.

FIG. 9 is a diagram of a system for integrating morphological characteristics and gene expression of a single cell according to an embodiment of the application.

FIG. 10 is an image of the cells separated from the microwells in Example 1 of the present application.

FIG. 11 is a diagram of cDNA library analysis via a bioanalyzer in Example 1 of the present application.

FIG. 12 is a diagram of the analysis of mouse cells and human cells in Example 1 of the present application.

### DETAILED DESCRIPTION

Unless otherwise defined, all technical and scientific terms used herein have the same meaning as commonly understood by those skilled persons in the art. The terms used herein is only for the purpose of describing specific implementation manners, and is not intended to limit the embodiments of the present application.

In the case of no conflict, the following embodiments and features in the embodiments can be combined with each other.

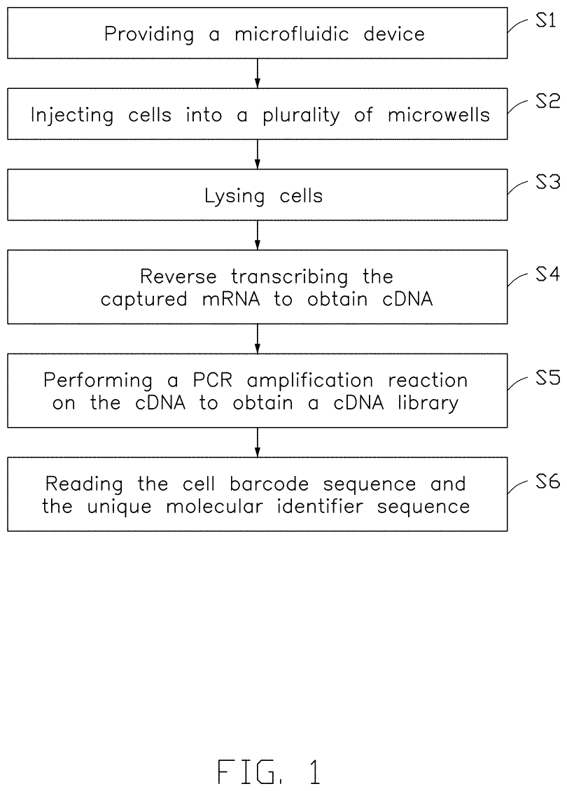

Referring to FIG. 1, an embodiment of the present application provides a method for integrating morphological characteristics and gene expression of individual cells, which comprises the following steps:

S1: a microfluidic device 10 is provided.

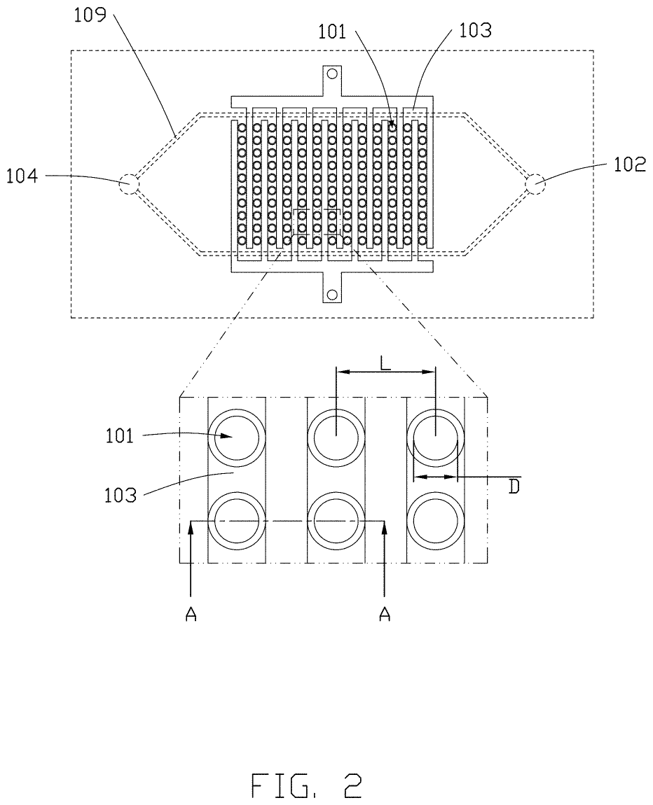

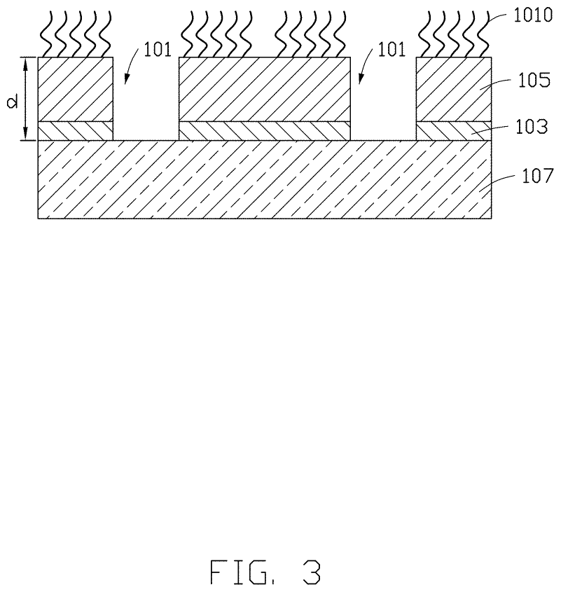

Referring to FIG. 2, in some embodiments, the microfluidic device 10 comprises a microwell array composed of a plurality of microwells 101 and an interdigital electrode 103. Referring to FIG. 3, in some embodiments, the microwell array can be fabricated on a SU-8 photoresist layer 105 by photolithography, and the SU-8 photoresist layer 105 is disposed on a glass substrate 107. In order to improve cell capturing efficiency, an interdigital electrode (IDE) 103 is patterned on the glass substrate 107 and under the microwell array, that is, the interdigital electrode 103 is arranged between the SU-8 photoresist layer 105 and the glass substrate 107. As shown in FIG. 2, the glass channel 109 is bonded onto the top of the SU-8 photoresist layer105 via an adhesive to form a sealed flow cell with a single inlet 102 and a single outlet 104. During the injection of a cell sample, a dielectrophoresis force (DEP force) generated by the interdigital electrode 103 will trap cells and guide them into the microwells 101.

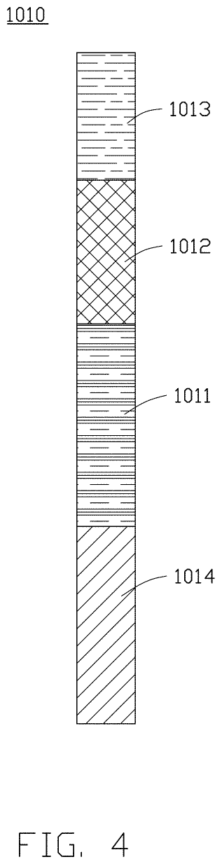

Referring to FIG. 3 and FIG. 4, in some embodiments, each microwell 101 comprises a plurality of capture oligonucleotides 1010, and each capture oligonucleotide 1010 comprises a known cell barcode sequence 1011, a unique molecule identifier (UMI) sequence 1012, and a capture sequence 1013. Furthermore, the capture oligonucleotide 1010 also comprises a PCR handle sequence 1014. There is a unique correspondence between the cell barcode sequence 1011 and the microwell 101 where the cell barcode sequence 1011 is located, and the cell barcode sequence 1011 is used to mark the microwell 101 where the cell barcode sequence 1011 is located, so as to mark the cell in the microwell 1011. The unique molecular identifier sequence 1012 is used to label the captured mRNA to avoid repeated counting after PCR. In some embodiments, the capture sequence 1013 may be a Poly dT sequence with a size of 30 bases, which is used to hybridize with the Poly dA segment of the mRNA released after cell lysis to capture the mRNA. In some embodiments, the cell barcode sequence 1011 comprises 6 to 10 bases. In some embodiments, the unique molecular identifier sequence 1012 comprises 7 to 15 bases. In some embodiments, the PCR handle sequence 1014 comprises 30 to 35 bases. Furthermore, the PCR handle sequence 1014 may include 32 bases.

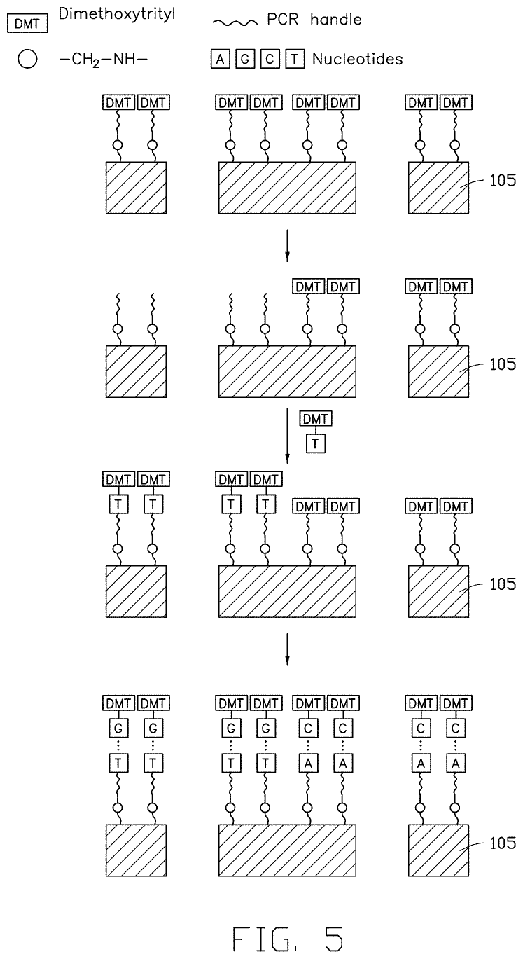

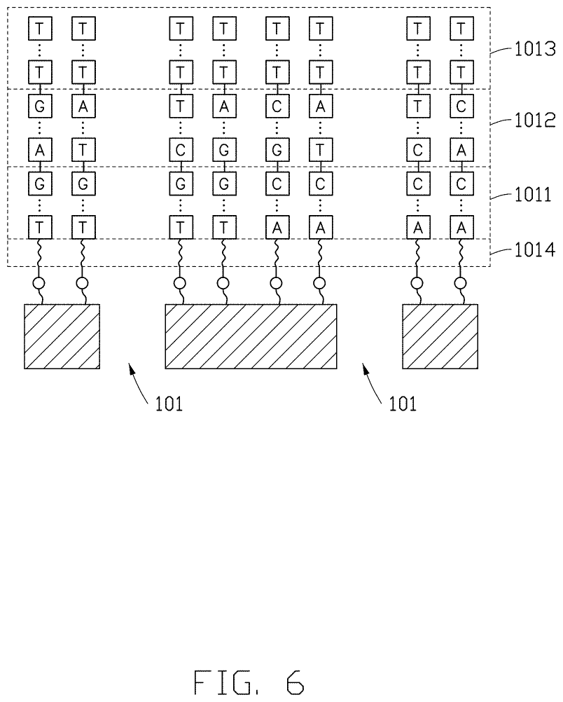

In some embodiments, the capture oligonucleotide 1010 can be synthesized by an inkjet printing technology, and the printing process is shown in FIG. 5. The epoxide groups on the SU-8 surface are available for direct conjugation of amine-modified oligonucleotides. First, the PCR handle sequence 1014 with 3′-dimethoxytrityl (DMT) protection is printed to the substrate, with the amine-modified 5′ end being linked to the surface of the SU-8 photoresist layer 105. Then, in order to generate the cell barcode sequence 1011, DMTs are selectively removed by chemicals, followed by a coupling reaction with specific nucleotides which only occurs on the spots that are de-protected. By repeating this process, a pre-designed 7-mers unique cell barcode sequence 1011 is developed for each microwell 101. Next, in order to generate a unique molecular identifier (UMI) sequence, a mixture of 4 types of DMT-protected nucleotides (A, G, C, T) is repeatedly added to all spots to generate 10-mers random unique sequences within each spot. The function of UMIs is to assign each captured mRNA transcript an identifier to avoid repeated counting after PCR. Finally, a poly dT tail is linked to each oligonucleotide, and the capture oligonucleotide 1010 as shown in FIG. 6 is obtained. The cell barcodes (7-mers) and UMIs (10-mers) have a maximum of 16,384 (4⁷) and 1,048,576 (4¹⁰) different sequences, that is, up to 16,384 different cells can be distinguished at a time. For samples with more than 16384 cells, the length of cell barcode sequence can be further increased.

To validate that the cell barcode sequence 1011 are well synthesized, 100 spots with known oligonucleotide sequence are distributed at different locations of the microwell array for hybridization with fluorescent probes. The hybridization rate of the 100 spots and the CV (coefficient of variation) of fluorescent signal intensity are characterized by fluorescence imaging. In the present application, the probe hybridization rate is greater than 95%, and CV of fluorescent signal intensity is less than 10%, indicating that the cell barcode sequence 1011 synthesized by this application has a high accuracy rate.

S2: cells are injected into the microwells 101, and the interdigital electrode 103 is used to capture a single cell in the microwell 101. The morphological characteristics of the cells in the microwells 101 are recorded for morphological analysis.

During operations, cells are injected through the inlet 102, which flow into the microwell array through the glass channel 109. The dielectrophoretic (DEP) force generated by the interdigital electrode 103 can trap single cells above the microwells 101. After the cells stop flowing, the interdigital electrode 103 is turned off, allowing the cells to be precipitated into the microwells 101. The excess cells outside the microwells 101 are washed away from the outlet 104. The ideal situation is that every microwell 101 traps only one cell. In order to achieve the ideal situation, the key parameters, including but not limited to microwell 101 dimension (diameter and depth), DEP force intensity, channel height, input cell concentration, and flow rate, are optimized to maximize the single-cell purity and microwell occupancy rate. Referring to FIG. 2 and FIG. 3, in one embodiment, the diameter (D) of the microwell 101 is 25 μm, the depth (d) of the microwell 101 is 20 μm, the distance (L) between two adjacent microwells 101 (the distance from the center to the center) is 85 μm, and the number of microwells 101 is 10,000 to 100,000.

After individual cells are trapped in the microwell array, the bright-field and fluorescent images of each cell for morphological profiling are recorded via a CCD (Charge Coupled Device) camera connected to a microscope. Meanwhile, the cell barcodes sequence 1011 on the capture oligonucleotide 1010 are assigned to the cells based on their locations in the array. For example, the cell barcode sequence of the microwell 101 in the first row and the first column is known as TACGAGC (TACGAGC is unique among all cell barcode sequences), and TACGAGC is assigned to the cell located in the first row and the first column of the microwells 101.

Air bubbles in the flow cell will cause failure to reverser transcription and PCR due to the bubble expansion at high temperature. To remove the air bubbles, the glass channel 107 is treated with 80% ethanol before buffer injection. Meanwhile, by applying a voltage to the interdigital electrode 103, the electrowetting behavior makes the surface hydrophilic, which is helpful to remove the bubbles trapped in the microwells 101.

S3: cells are lysed so that the mRNA released by the cell is captured by the capture oligonucleotide in the microwell 101 where the cell is located.

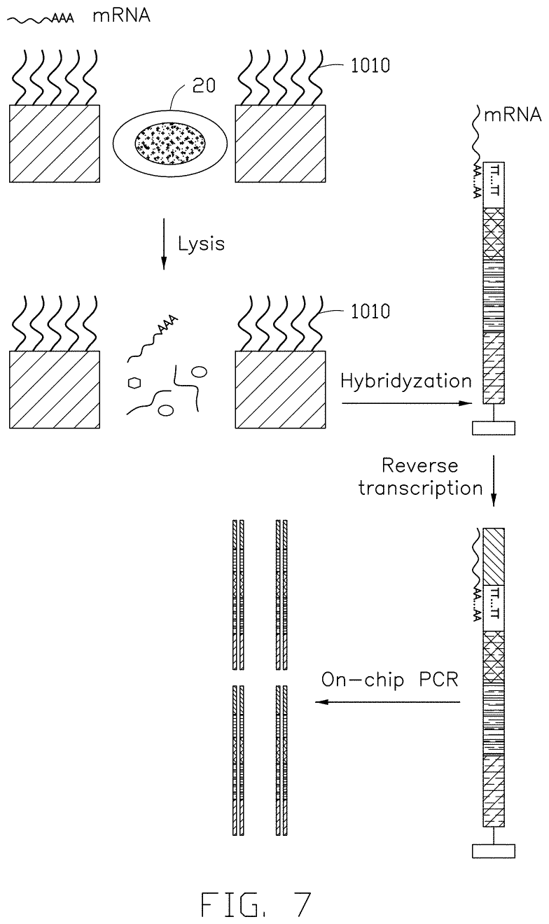

Referring to FIG. 7, after cell imaging, in-site cell lysis is carried out via the injection of lysis buffer. The mRNA (with a poly dA tail) released by cells will be captured by the nearest capture oligonucleotide 1010 due to hybridization between poly dA and poly dT tails (i.e., capture sequence 1013 in the capture oligonucleotide 1010).

S4: the captured mRNA is reverse transcribed to obtain cDNA, each cDNA comprises a capture oligonucleotide sequence 1010 and a nucleotide sequence complementary to the captured mRNA.

Referring to FIG. 7, after sample wash and injection of reverse transcriptase reagents, the mRNA sequence is transferred to the capture oligonucleotide 1010 via reverse transcription with template switching to obtain a cDNA with a sequence complementary to the mRNA. The cDNA also contains the entire sequence of the capture oligonucleotide 1010, that is, the cDNA also contains the cell barcode sequence 1011, the unique molecular identifier sequence 1012, the capture sequence 1013, and the PCR handle sequence 1014.

S5: perform a PCR amplification reaction on the cDNA to obtain a cDNA library, and the cDNA library is sequenced.

Referring to FIG. 7, after digesting excess barcoded oligonucleotides that did not capture an mRNA using exonuclease enzyme I, on-chip PCR amplification of the cDNA is performed. The reagents and specific steps used for on-chip PCR amplification are common knowledge in the art, and will not be repeated here. After on-chip PCR amplification, high-throughput sequencing of the cDNA library is performed on a DNA sequencer to read the sequence information of the cDNA library.

S6: the cell barcode sequence 1011 and the unique molecular identifier sequence 1012 are read according to a sequencing result, and the morphological characteristics and gene expression information of the cell in the microwell 101 are integrated together.

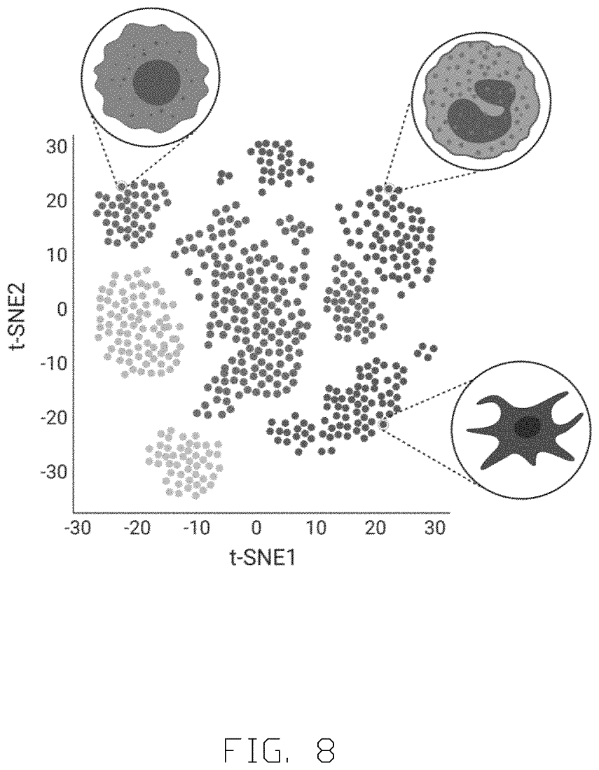

Specifically, after identifying the genes, reads are organized by their cell barcode sequence 1011 and individual UMIs are counted for each gene in each cell. By reading the cell barcode sequence 1011 in the sequencing result, the microwell 101 can be located, and the morphological characteristics (the bright-field image and the fluorescence image obtained in step S2) of the cell corresponding to the microwell 101 can be obtained. Furthermore, the morphological characteristics and gene expression of the cell are integrated by a controller which includes an analysis software. In some embodiments, the analysis software is t-distributed stochastic neighbor embedding (tSNE), a data visualization tool that can reduce high-dimensional data to two-dimensional or three-dimensional, and then draw it into a graph. Referring to FIG. 8, the single-cell expression profiles are eventually be plotted two-dimensionally (tSNE) for visualized analysis and integrated with their morphological features. It is understandable that other analysis software or methods can also be used to analyze the morphological characteristics and gene expression profiles of single cells.

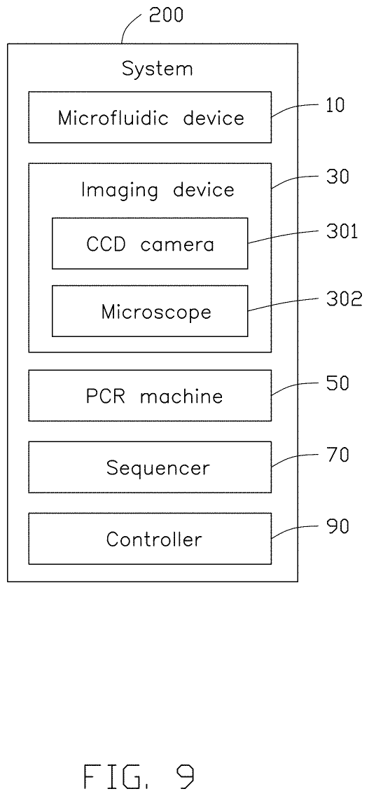

Referring to FIG. 9, the present application also provides a system 200 that integrates the morphological characteristics and gene expression of single-cell. The system 200 comprises a microfluidic device 10, an imaging device 30, a PCR machine 50, a sequencer 70, and a controller 90. The microfluidic device 10 is used to disperse the cell population into individual cells and mark the individual cell. The microfluidic device 10 comprises a microwell array composed of a plurality of microwells 101 and an interdigital electrode 103. Each microwell 101 comprises a plurality of capture oligonucleotides 1010, and each capture oligonucleotide 1010 comprises a known cell barcode sequence 1011, a unique molecular identifier sequence 1012, and a capture sequence 1013. There is a unique correspondence between the cell barcode sequence 1011 and the microwell 101 where it is located, and the cell barcode sequence 1011 is used to mark the cell in the microwell 101. The unique molecular identifier sequence 1012 is used to label the captured mRNA. The imaging device 30 is used to capture images the cells 20 and record the morphological characteristics of the cells 20 for morphological analysis. In some embodiments, the imaging device 30 comprises a CCD camera 301 and a microscope 302 connected to the CCD camera 301. The PCR machine 50 is used for PCR amplification of cDNA to obtain a cDNA library. The sequencer 70 is used to sequence the cDNA library. The controller 90 is used to integrate the morphological characteristics and gene expression of the cells 20. In some embodiments, the controller includes an analysis software, and the analysis software comprises but is not limited to tSNE.

The application will be further described below in conjunction with specific embodiments.

### EXAMPLE 1

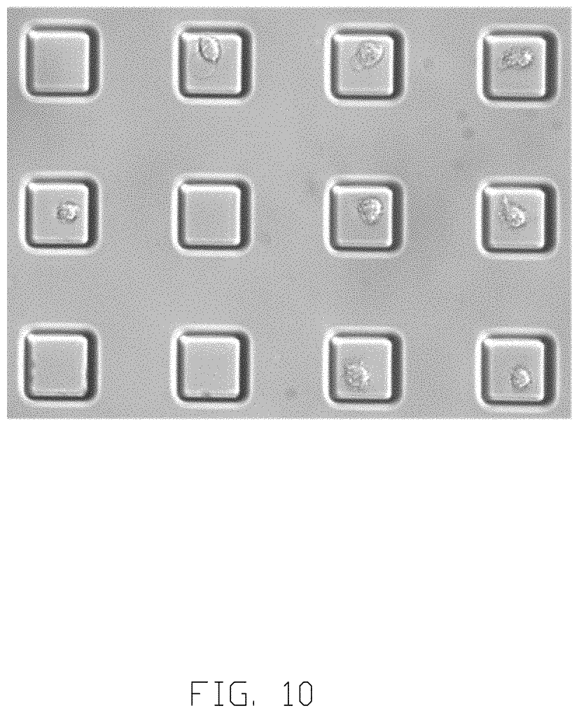

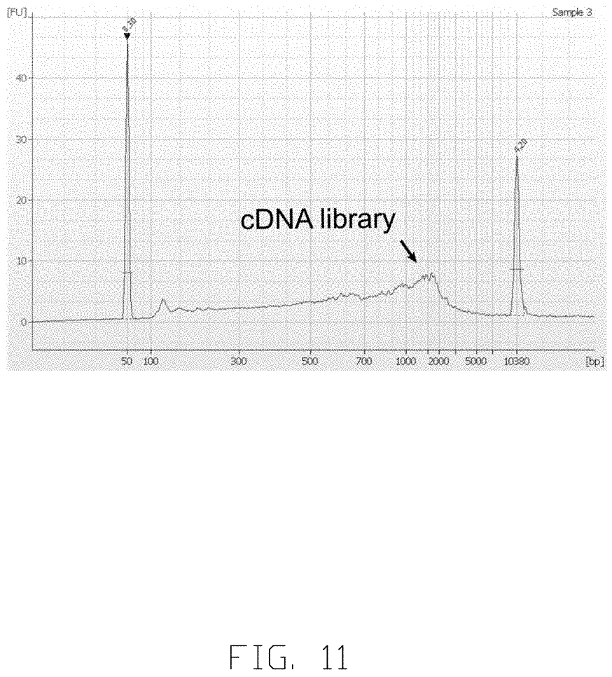

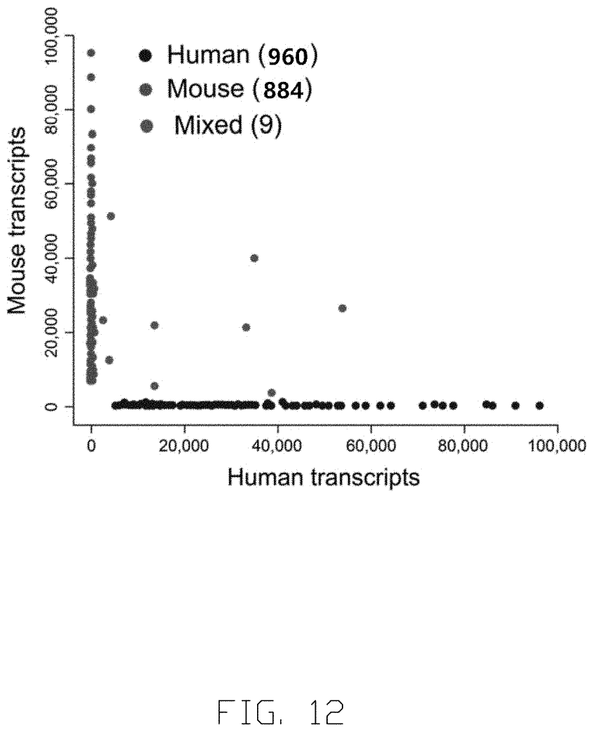

HEK 293T cells (human embryonic kidney cell line) and mouse 3T3 cell lines were mixed at the same concentration, and the mixture was analyzed for single-cell morphological characteristics and gene expression profiles. Individual cells were isolated in the microwells 101 for imaging, as shown in FIG. 10. After cell lysis, reversed transcription, and on-chip PCR amplification on device, the cDNA library was purified using SPRI beads and measured by Agilent 2100 Bioanalyzer (DNA 7500 Kit) as shown in FIG. 11. It can be seen from FIG. 11 that the average size of the cDNA was between 1000-2000 bp. The cDNA library was sequenced using a MiSeq sequencer, and the number of individual UMIs was plotted in FIG. 12. As can be seen from FIG. 12, 960 human-only transcripts, 884 mouse-only transcripts and 9 human-mouse mixed transcripts are collected, indicating a multiplet rate of 0.4%, which demonstrates a good performance of the single-cell isolation.

If cells from two cell lines are isolated into the same microwell 101 and their mRNAs are captured by the same capture oligonucleotide 1010, the genes from the two species will share the same cell barcode sequence 1011, the proportion of which reveals the single-cell purity. The single-cell purity could be further improved by analyzing the cell images to screen out the cell doublets and multiplets. In this example, the single-cell purity is greater than 95%.

The recovery rate is calculated as the percentage of recovered cells (number of cell barcode sequences) to the total input cell number. In this example, the recovery rate is greater than 80%.

The contamination of ambient RNA from original biofluids or cell disruption decreases the accuracy of interpretation. Synthetic RNA controls are spiked into the sample with known concentration and sequence to evaluate the RNA contamination, which is defined as the percentage of recovered spiked RNAs (number of individual UMIs with spike-RNA sequence) to the total input spiked RNA amount. In this example, RNA contamination is less than 5%.

To characterize the detection sensitivity, mouse cells are spiked into human cells in different ratios of concentration ranging from 1:1 (50%) to 1:99 (1%). The sensitivity is determined as the percentage above which the mouse cells can be detected. In this example, the sensitivity is less than 5%, that is, the lower limit of detection of mouse cell concentration is 5%, and mouse cell can be detected as long as the concentration is greater than 5%.

In this application, a plurality of individual cells are placed in a plurality of microwells 101 in the microfluidic device, and from the beginning (imaging) to the end (sequencing), each cell will be assigned a unique known cell barcode sequence to observe their phenotype before processing for sequencing. The cell barcode sequence in the capture oligonucleotide can be “read” in the microwells 101 and also can be “read” from the sequence reads obtained from the cDNA library, thereby the genome/transcriptome data (mRNA sequence information) is linked to the observed phenotype of single-cell, so that the morphological phenotype is directly related to gene expression. This method focuses on integrating the morphological characteristics and gene expression profiles of isolated single cells. It has the characteristics of high efficiency, single-cell purity (greater than 95%), recovery rate (greater than 80%) and sensitivity (less than 5%), and low RNA contamination (less than 5%). It can facilitate fundamental biological studies, develop multi-dimensional biomarker signatures for diseases, and accelerate drug discovery and development.

The above descriptions are some specific implementation manners of the present application, but in the actual application process, they should not be limited to these implementation manners. For persons skilled in the art, other modifications and changes made according to the technical concept of this application should all belong to the protection scope of this application.

What is claimed is:

1. A method for integrating morphological characteristics and gene expression of individual cells, comprising:
providing a microfluidic device, wherein the microfluidic device comprises a microwell array composed of a plurality of microwells and an interdigital electrode, each of the plurality of microwells comprises a plurality of capture oligonucleotides, each of the plurality of capture oligonucleotide comprises a cell barcode sequence, a unique molecular identifier sequence, and a capture sequence, each cell barcode sequence corresponds to one microwell in which the cell barcode sequence is located, the cell barcode sequence is configured to mark a cell in the microwell in which the cell barcode sequence is located, and the unique molecular identifier sequence is configured to mark mRNA captured by the capture oligonucleotide;
injecting cells into the plurality of microwells, using the interdigital electrode to capture a single cell above each of the plurality of microwells, and recording the morphological characteristics of the cell in the plurality of microwell;
lysing cells so that the mRNA released by the cell is captured by the plurality of capture oligonucleotide in the plurality of microwell where the cell is located;
reverse transcribing the captured mRNA to obtain cDNA, wherein the cDNA comprises a capture oligonucleotide sequence and a nucleotide sequence complementary to the captured mRNA;
performing a PCR amplification reaction on the cDNA to obtain a cDNA library, and sequencing the cDNA library;
reading the cell barcode sequence and the unique molecular identifier sequence according to a sequencing result, and the morphological characteristics and gene expression of the cell in the microwell are integrated together.

2. The method of claim 1, wherein the cell barcode sequence comprises 6 to 10 bases.

3. The method of claim 1, wherein the unique molecular identifier sequence comprises 7 to 15 bases.

4. The method of claim 1, wherein the plurality of capture oligonucleotide further comprises a PCR handle sequence, and the PCR handle sequence comprises 30-35 bases.

5. The method of claim 1, wherein the morphological characteristics of each cell comprise bright-field images and fluorescent images.

6. A system for integrating morphological characteristics and gene expression of individual cells, comprising:
a microfluidic device configured to disperse a cell population into individual cells and mark the individual cells, wherein the microfluidic device comprises a microwell array composed of a plurality of microwells and an interdigital electrode, each of the plurality of microwell comprises a plurality of capture oligonucleotides, and each of the plurality of capture oligonucleotides comprises a known cell barcode sequence, a unique molecular identifier sequence, and a capture sequence, the cell barcode sequence has a unique corresponding relationship with the microwell in which it is located, the cell barcode is used to mark the individual cells in the plurality of microwell, and the unique molecular identifier sequence is used to mark the captured mRNA;
an imaging device configured to capture images of the individual cells and record the morphological characteristics of the individual cells;
a PCR machine configured to perform an PCR amplification reaction on cDNA to obtain a cDNA library;
a sequencer configured to sequence the cDNA library;
a controller configured to integrate the morphological characteristics and gene expression of the individual cells.

7. The system of claim 6, wherein a diameter of the plurality of microwells is 25 μm, a depth of the plurality of microwells is 20 μm, and a distance between two adjacent microwells is 85 μm.

8. The system of claim 6, wherein the number of the plurality of microwells is 10,000 to 100,000.

9. The system of claim 6, wherein the interdigital electrode is disposed under the microwell array.

10. The system of claim 6, wherein the imaging device comprise a CCD camera and a microscope connected to the CCD camera.
